(19) United States
(12) Patent Application Publication
(10) Pub. No.: US 2010/0030002 A1
(43) Pub. Date: Feb. 4, 2010

(54) Ethylene production from acetic acid utilizing dual reaction zone process

(76) Inventors: Victor J. Johnston, Houston, TX (US); James H. Zink, League City, TX (US); Josefina T. Chapman, Houston, TX (US); Laiyuan Chen, Houston, TX (US); Barbara F. Kimmich, League City, TX (US)

Correspondence Address: FERRELLS, PLLC, P. O. BOX 312, CLIFTON, VA, 20124-1706, US

(21) Appl. No.: 12/221,138

(22) Filed: Jul. 31, 2008

(51) Int. Cl. C07C 1/20

(52) U.S. Cl. 585/638

(57) ABSTRACT

A process for selective formation of ethylene from acetic acid includes contacting a feed stream containing acetic acid and hydrogen at an elevated temperature with a first catalytic composition including a suitable hydrogenating catalyst in a first reaction zone to form an intermediate mixture including ethanol and ethyl acetate; and subsequently reacting the intermediate mixture over a suitable dehydrating and/or cracking catalyst in a second reaction zone to form ethylene. Selectivities of ethylene of over 80% are achieved.

### FIELD OF THE INVENTION

The present invention relates generally to a process for the production of ethylene from acetic acid. More specifically, the present invention relates to a process including hydrogenating acetic acid utilizing a first catalyst composition in a first reaction zone and dehydrating or cracking hydrogenated intermediates with a second catalyst in a second reaction zone to form ethylene with high selectivity.

### BACKGROUND

There is a long felt need for an economically viable process to convert acetic acid to ethylene. Ethylene is an important commodity feedstock for a variety of industrial products; for example, ethylene can then be converted to a variety of polymer and monomer products. Fluctuating natural gas and crude oil prices contribute to fluctuations in the cost of conventionally produced, petroleum or natural gas-sourced ethylene, making the need for alternative sources of ethylene all the greater when oil prices rise.

It has been reported that ethylene can be produced from various ethyl esters in the gas phase in the temperature range of 150-300° C. over zeolite catalysts. The types of ethyl esters that can be employed include ethyl esters of formic acid, acetic acid and propionic acid. See, for example, U.S. Pat. No. 4,620,050 to Cognion et al., where selectivity is reported to be acceptable.

U.S. Pat. No. 4,270,015 to Knifton describes obtaining ethylene involving a two-step process in which a mixture of carbon monoxide and hydrogen (commonly known as synthesis gas) is reacted with a carboxylic acid containing 2 to 4 carbon atoms to form the corresponding ethyl ester of said carboxylic acid which is subsequently pyrolyzed in a quartz reactor at elevated temperatures in the range of about 200° to 600° C. to obtain ethylene. The ethylene thus produced contains other hydrocarbons, particularly, ethane as an impurity. It was also reported therein that the concentration of ethane can reach high values, near 5% by pyrolyzing pure ethyl propionate at 460° C. More importantly, the conversion of the esters and yield of ethylene are reported to be very low.

U.S. Pat. No. 4,399,305 to Schreck describes obtaining high purity ethylene from ethyl acetate employing a cracking catalyst composed of a perfluorosulfonic acid resin commercially sold under the trademark NAFION® by E.I. DuPont de Nemours & Co.

On the other hand, Malinowski et al. Bull. Soc. Chim. Belg. (1985), 94(2), 93-5, disclose that reaction of substrates such as acetic acid on low-valent titanium heterogenized on support materials such as silica (SiO₂) or titania (TiO₂) resulted in a mixture of products including diethyl ether, ethylene and methane where selectivity is poor.

WO 2003/040037 discloses that crystalline microporous metalloalumino-phosphates (ELAPO), particularly, SAPO-type zeolites, such as SAPO-5, SAPO-11, SAPO-20, SAPO-18 and SAPO-34, having Si/Al ratio of 0.03-017 are useful as adsorbent or as a catalyst for the production of olefins from an oxygenated feedstock containing methanol, ethanol, n-propanol, isopropanol, C4-C20 alcohols, methyl ethyl ether, di-methyl ether, di-ethyl ether, di-isopropyl ether, formaldehyde, dimethyl carbonate, dimethyl ketone and/or acetic acid. A similar disclosure utilizes a silicoaluminophosphate molecular sieves comprising at least one intergrown phase of molecular sieve. It is reported that in this process a feedstock containing an oxygenate contacts a catalyst comprising the molecular sieve in a reaction zone of a reactor at conditions effective to produce light olefins, particularly ethylene and propylene. See U.S. Pat. No. 6,812,372 to Janssen et al. It is mentioned that such oxygenated feedstocks include acetic acid, but the disclosure appears to be limited to either methanol or dimethyl ether. See, also, U.S. Pat. No. 6,509,290 to Vaughn et al., which further discloses conversion of oxygenated feedstocks to olefins.

Bimetallic ruthenium-tin/silica catalysts have been prepared by reaction of tetrabutyl tin with ruthenium dioxide supported on silica. It has been reported that these catalysts exhibit different selectivities based on their content of tin/ruthenium ratio (Sn/Ru). Specifically it has been reported that the selectivity for the hydrogenolysis of ethyl acetate is quite different, which depends upon the Sn/Ru ratio in the catalyst. For instance, with ruthenium alone on SiO₂, the reaction is not selective: methane, ethane, carbon monoxide, carbon dioxide as well as ethanol and acetic acid are produced. Whereas, with low tin content, it has been reported that the catalysts are fairly selective for the formation of acetic acid, while at higher Sn/Ru ratios, ethanol is the only detected product. See Loessard et al., Studies in Surface Science and Catalysis (1989), Volume Date 1988, 48 (Struct. React. Surf.), 591-600.

The catalytic reduction of acetic acid has also been studied. For instance, Hindermann et al., J. Chem. Res., Synopses (1980), (11), 373, have disclosed the catalytic reduction of acetic acid on iron and on alkali-promoted iron. In their study they found that the reduction of acetic acid on alkali-promoted iron, followed at least two different routes depending on the temperature. For example, they found that at 350° C., the Piria reaction was predominant and gave acetone and carbon dioxide, as well as they observed decomposition products methane and carbon dioxide, whereas the decomposition products were reduced at lower temperatures. On the other hand, at 300° C. a normal reduction reaction was observed resulting in the formation of acetaldehyde and ethanol.

From the foregoing it is apparent that existing processes do not have the requisite selectivity to ethylene or existing art specifies starting materials other than acetic acid, which are expensive and/or intended to produce products other than ethylene.

### SUMMARY OF THE INVENTION

A process for selective formation of ethylene from acetic acid includes: contacting a feed stream containing acetic acid and hydrogen at an elevated temperature with a first catalytic composition including a suitable hydrogenating catalyst in a first reaction zone to form an intermediate mixture preferably including acetic acid, ethanol and ethyl acetate; and subsequently reacting said hydrogenated mixture over a suitable dehydrating and/or cracking catalyst in a second reaction zone to form ethylene.

### BRIEF DESCRIPTION OF DRAWING

The invention is described in detail below with reference to the single FIGURE which is a schematic diagram of a layered fix bed reactor.

### DETAILED DESCRIPTION OF THE INVENTION

The invention is described in detail below with reference to numerous embodiments for purposes of exemplification and illustration only. Modifications to particular embodiments within the spirit and scope of the present invention, set forth in the appended claims, will be readily apparent to those of skill in the art.

Unless more specifically defined below, terminology as used herein is given its ordinary meaning. % and like terms refer to mole percent unless otherwise indicated.

“Conversion” is expressed as a mole percentage based on acetic acid in the feed.

“Selectivity” is expressed as a mole percent based on converted acetic acid. For example, if the conversion is 50 mole % and 50 mole % of the converted acetic acid is converted to ethylene, we refer to the ethylene selectivity as 50%. Ethylene selectivity is calculated from gas chromatography (GC) data as follows:

$$Ethylene Selectivity , \% = 100 * \frac{mmol Ethylene out ( G C )}{\frac{Total mmol C out ( G C )}{2} - mmol AcOH out ( G C )}$$

Without intending to be bound by theory, it is believed the conversion of acetic acid to ethylene in accordance with the invention proceeds in accordance with one or more of the following chemical equations:

### Step 1a: Hydrogenation of Acetic Acid to Ethanol.

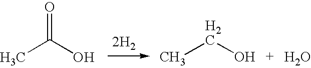

### Step 1b: Hydrogenation of Acetic Acid to Ethyl Acetate

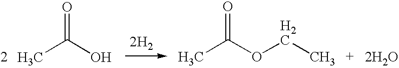

### Step 1c: Cracking of Ethyl Acetate to Ethylene and Acetic Acid

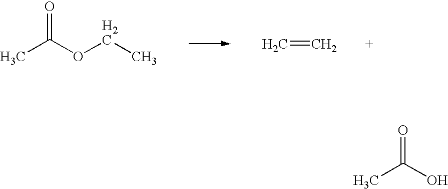

### Step 2a: Dehydration of Ethanol to Ethylene.

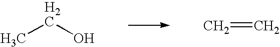

The process of the invention may be practiced in a variety of configurations using a fixed bed reactor or a fluidized bed reactor as one of skill in the art will readily appreciate. An adiabatic reactor could be used, or a shell and tube reactor provided with a heat transfer medium could be used. In any case, the two reaction zones may be housed in a single vessel with different layers in a fixed bed reactor or the two reaction zones may be housed in single vessel fluidized bed system with baffles and separators providing two distinct zones. Alternatively, two vessels could be used to house the different reaction zones. In any case, multiple reactors having two zones may be run in a parallel, for example, multiple tubular reactors having layered fixed beds arranged in parallel may be used if convenient.

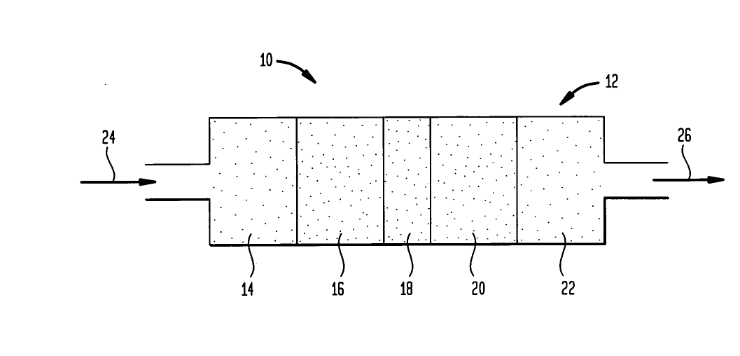

There is shown schematically in FIG. 1 a tubular reactor with a layered fixed bed 10. Bed 10 is a fixed bed in a vessel 12 which includes a layer of inert particulate material making up a mixing zone or layer 14, a first reaction zone or layer 16, an optional separator zone or layer 18, a second reaction zone or layer 20 and a spacer zone or layer 22. A reaction mixture including acetic acid, hydrogen and optionally an inert carrier gas is fed to bed 10 as a stream 24 under pressure to mixing zone 14. The stream is subsequently supplied (by way of pressure drop) to first reaction zone or layer 16. Reaction zone 16 comprises a first catalytic composition including a suitable hydrogenating catalyst where hydrogenated acetic acid intermediates are produced. Suitably, the first catalytic composition is in particulate form.

After hydrogenation, the mixture moves forward through optional separator zone 18 to second reaction zone or layer 20 containing a second catalytic composition comprising a suitable dehydration and/or cracking catalyst.

In zone 20, the hydrogenated acetic acid intermediates such as ethyl acetate and ethanol are dehydrated and/or cracked to produce ethylene and the product is forwarded to spacer zone 22, ultimately exiting bed 10 as product stream 26 at a pressure less than the inlet pressure to vessel 12.

Layers 14, 18 and 22 are optional and may be formed of inert particulate material of suitable size in the configuration shown in FIG. 1. In other layouts or configurations, equivalent means may be of any suitable design effective to promote mixing, separation, heat transfer and so forth as will be appreciated by one of skill in the art.

Various hydrogenation catalysts known to one skilled in the art can be employed in hydrogenating acetic acid to ethanol in the first step of the process of this invention. The hydrogenation catalysts that are suitable are the ones which are metal catalysts on a suitable support. As examples of such catalysts the following catalysts may be mentioned without any limitation: copper, nickel, aluminum, chromium, zinc, and a mixture thereof. Typically, either a single metal or a bimetallic catalyst on a suitable support can be used as a hydrogenation catalyst. Thus either copper alone or in combination with aluminum, chromium or zinc are particularly preferred.

Various catalyst supports known in the art can be used to support the catalysts of this invention. Examples of such supports include without any limitation, iron oxide, silica, alumina, titania, zirconia, magnesium oxide, calcium silicate, carbon, graphite and a mixture thereof.

In an embodiment of this invention, specific examples of supported hydrogenation catalysts include iron oxide, silica, alumina, titania, zirconia, magnesium oxide, calcium silicate, carbon, graphite and a mixture thereof. Particularly, as noted above, copper supported on iron oxide and copper-aluminum catalysts are preferred.

A few of the commercially available catalysts include the following: copper-aluminum catalyst sold under the name of T-4489 by Sud Chemie; copper-zinc catalysts sold under the name of T-2130, T-4427 and T-4492; copper-chromium catalysts sold under the name of T-4419 and G-99B; and nickel catalysts sold under the name of NiSAT 310, C47-7-04, G-49, and G-69; all sold by Sud Chemie. Copper-aluminum catalyst sold under the name of T-4489 is particularly preferred.

The amount of metal loading on a support is not very critical in this invention and can vary in the range of about 3 weight percent to about 10 weight percent. A metal loading of about 4 weight percent to about 6 weight percent based on the weight of the support is particularly preferred. Thus, for example 4 to 6 weight percent of copper supported on iron oxide is particularly a preferred catalyst.

The metal impregnation can be carried out using any of the known methods in the art. Typically, before impregnation the supports are dried at 120° C. and shaped to particles having size distribution in the range of about 0.2 to 0.4 mm. Optionally the supports may be pressed, crushed and sieved to a desired size distribution. Any of the known methods to shape the support materials into desired size distribution can be employed.

For supports having low surface area, such as for example alpha-alumina or iron oxide, the metal solutions are added in excess until complete wetness or excess liquid impregnation so as to obtain desirable metal loadings.

As noted above, a few of the hydrogenation catalysts are bimetallic. Generally, in such cases, one metal acts as a promoter metal and the other metal is the main metal. For instance copper, nickel, cobalt and iron are considered to be main metals for preparing hydrogenation catalysts of this invention. The main metal can be combined with a promoter metal such as tungsten, vanadium, molybdenum, chromium or zinc. However, it should be noted that sometimes main metal can also act as a promoter metal or vice versa. For example, nickel can be used as a promoter metal when iron is used as a main metal. Similarly, chromium can be used as a main metal in conjunction with copper (i.e., Cu—Cr as main bimetallic metals), which can further be combined with promoter metals such as cerium, magnesium or zinc.

The bimetallic catalysts are generally impregnated in two steps. First, the “promoter” metal is added, followed by “main” metal. Each impregnation step is followed by drying and calcination. The bimetallic catalysts may also be prepared by co-impregnation. In the case of trimetallic Cu/Cr-containing catalysts as described above, a sequential impregnation may be used, starting with the addition of the “promoter” metal. The second impregnation step may involve co-impregnation of the two principal metals, i.e., Cu and Cr. For example, Cu—Cr—Ce on SiO₂ may be prepared by a first impregnation of Cerium nitrate, followed by the co-impregnation of copper and chromium nitrates. Again, each impregnation is followed by drying and calcinations. In most cases, the impregnation may be carried out using metal nitrate solutions. However, various other soluble salts which upon calcination releases metal ions can also be used. Examples of other suitable metal salts for impregnation include metal hydroxide, metal oxide, metal acetate, ammonium metal oxide, such as ammonium heptamolybdate hexahydrate, metal acids, such as perrhenic acid solution, metal oxalate, and the like.

In another aspect of the process of this invention, any of known dehydration catalysts can be employed in the second step of the process of this invention. Typically, a zeolite catalyst is employed as a dehydration catalyst. While any zeolite having a pore diameter of at least about 0.6 nm can be used, preferably employed among such zeolites are the dehydration catalyst selected from the group consisting of mordenites, ZSM-5, a zeolite X and a zeolite Y.

The preparation of large-pore mordenites is described, for example, in U.S. Pat. No. 4,018,514 and in Mol. Sieves Pap. Conf., 1967, 78, Soc. Chem. Ind. London, by D. DOMINE and J. QUOBEX.

Zeolite X is described, for example, in U.S. Pat. No. 2,882,244 and zeolite Y in U.S. Pat. No. 3,130,007.

Various zeolites and zeolite-type materials are known in the art for the catalysis of chemical reactions. For example, U.S. Pat. No. 3,702,886, of Argauer, discloses a class of synthetic zeolites, characterized as “Zeolite ZSM-5”, which are effective for the catalysis of various hydrocarbon conversion processes.

The zeolites suitable for the procedure of the invention can be in the basic form, in the partially or totally acidified form, or in the partially dealuminated form.

The active catalyst in the process of the present invention, characterized as “H-ZSM-5” or “H-mordenite” zeolites are prepared from a corresponding “ZSM-5” zeolite or “mordenite” zeolite by replacing most, and generally at least about 80% of the cations of the latter zeolite with hydrogen ions using techniques well-known in the art. These zeolite catalysts are essentially crystalline aluminosilicates or in the neutral form a combination of silica and alumina in a well defined crystalline structure. In a particularly preferred class of zeolite catalysts for purposes of the present invention, the molar ratio of SiO₂ to Al₂O₃ in these zeolites is within the ratio of about 10 to 60.

As noted earlier, ethylene is produced by dehydration as well as the decomposition or “cracking” of ethyl acetate to ethylene and acetic acid. This may be a catalyzed reaction if so desired, utilizing a cracking catalyst. Suitable cracking catalysts include sulfonic acid resins such as perfluorosulfonic acid resins disclosed in U.S. Pat. No. 4,399,305, noted above, the disclosure of which is incorporated herein by reference. Zeolites are also suitable as cracking catalysts as noted in U.S. Pat. No. 4,620,050, the disclosure of which is also incorporated herein by reference. Thus, a zeolite catalyst may be used to concurrently dehydrate ethanol to ethylene and decompose ethyl acetate to ethylene in a highly efficient process of the invention.

Selectivities of acetic acid to ethylene are suitably more than 10%, such as at least 20%, at least 40%, at least 60% or at least 80%. Depending on the by-product mix, it may be desirable to operate at intermediate selectivities, provided selectivity to undesirable products such as CO₂ remains low.

Preferably, for the purposes of the process of this invention, the suitable hydrogenation catalyst is either copper on iron oxide or copper-aluminum catalyst, sold under the tradename of T-4489 by Sud Chemie and the dehydration catalyst is H-mordenite. In this embodiment of the process of this invention, the copper loading on the iron oxide support or in the bimetallic copper-aluminum catalyst is typically in the range of about 3 weight percent to about 10 weight percent, preferably it is in the range of about 4 weight percent to about 6 weight percent.

In one of the embodiment of this invention, it is preferred that the hydrogenation and dehydration catalyst are layered. Preferably, the top layer of the catalyst bed is a hydrogenation catalyst and the bottom layer is dehydration catalyst.

In another aspect of the process of this invention, the hydrogenation and dehydration are carried out at a pressure just sufficient to overcome the pressure drop across the catalytic bed.

The reaction may be carried out in the vapor or liquid state under a wide variety of conditions. Preferably, the reaction is carried out in the vapor phase. Reaction temperatures may be employed, for example in the range of about 200° C. to about 375° C., preferably about 250° C. to about 350° C. The pressure is generally uncritical to the reaction and subatmospheric, atmospheric or superatmospheric pressures may be employed. In most cases, however, the pressure of the reaction will be in the range of about 1 to 30 atmospheres absolute.

Although the reaction consumes two moles of hydrogen per mole of acetic acid to produce a mole of ethanol, the actual molar ratio of acetic acid to hydrogen in the feed stream may be varied between wide limits, e.g. from about 100:1 to 1:100. It is preferred however that such ratio be in the range of about 1:20 to 1:2.

The raw materials used in connection with the process of this invention may be derived from any suitable source including natural gas, petroleum, coal, biomass and so forth. It is well known to produce acetic acid through methanol carbonylation, acetaldehyde oxidation, ethylene oxidation, oxidative fermentation, and anaerobic fermentation and so forth. As petroleum and natural gas have become more expensive, methods for producing acetic acid and intermediates such as methanol and carbon monoxide from alternate carbon sources have drawn more interest. Of particular interest is the production of acetic acid from synthesis gas (syngas) that may be derived from any suitable carbon source. U.S. Pat. No. 6,232,352 to Vidalin, the disclosure of which is incorporated herein by reference, for example, teaches a method of retrofitting a methanol plant for the manufacture of acetic acid. By retrofitting a methanol plant the large capital costs associated with CO generation for a new acetic acid plant are significantly reduced or largely eliminated. All or part of the syngas is diverted from the methanol synthesis loop and supplied to a separator unit to recover CO and hydrogen, which are then used to produce acetic acid. In addition to acetic acid, the process can also be used to make hydrogen which is utilized in connection with this invention.

U.S. Pat. No. RE 35,377 Steinberg et al., also incorporated herein by reference, provides a method for the production of methanol by conversion of carbonaceous materials such as oil, coal, natural gas and biomass materials. The process includes hydrogasification of solid and/or liquid carbonaceous materials to obtain a process gas which is steam pyrolized with additional natural gas to form synthesis gas. The syngas is converted to methanol which may be carbonylated to acetic acid. The method likewise produces hydrogen which may be used in connection with this invention as noted above. See also, U.S. Pat. No. 5,821,111 Grady et al., which discloses a process for converting waste biomass through gasification into synthesis gas as well as U.S. Pat. No. 6,685,754 Kindig et al., the disclosures of which are incorporated herein by reference.

The acetic acid may be vaporized at the reaction temperature, and then it can be fed along with hydrogen in undiluted state or diluted with a relatively inert carrier gas, such as nitrogen, argon, helium, carbon dioxide and the like.

Alternatively, acetic acid in vapor form may be taken directly as crude product from the flash vessel of a methanol carbonylation unit of the class described in U.S. Pat. No. 6,657,078 of Scates et al., the disclosure of which is incorporated herein by reference. The crude vapor product may be fed directly to the reaction zones of the present invention without the need for condensing the acetic acid and light ends or removing water, saving overall processing costs.

Contact or residence time can also vary widely, depending upon such variables as amount of acetic acid, catalyst, reactor, temperature and pressure. Typical contact times range from a fraction of a second to more than several hours when a catalyst system other than a fixed bed is used, with preferred contact times, at least for vapor phase reactions, between about 0.5 and 100 seconds.

Typically, the catalyst is employed in a fixed bed reactor e.g. in the shape of an elongated pipe or tube where the reactants, typically in the vapor form, are passed over or through the catalyst. Other reactors, such as fluid or ebullient bed reactors, can be employed, if desired. In some instances, it is advantageous to use the hydrogenation and zeolite catalysts in conjunction with an inert material such as glass wool to regulate the pressure drop of the reactant stream through the catalyst bed and the contact time of the reactant compounds with the catalyst particles.

In one of the preferred embodiments there is also provided a process for selective formation of ethylene from acetic acid comprising: contacting a feed stream of acetic acid and hydrogen at a temperature in the range of about 250° C. to 350° C. with a hydrogenation catalyst chosen from copper supported on iron oxide or copper-aluminum catalyst to form an intermediate mixture including acetic acid, ethanol and ethyl acetate; and concurrently reacting said mixture over a dehydrating catalyst chosen from H-mordenite zeolite or sodium Y zeolite to form ethylene.

In this embodiment of the process of this invention, the preferred hydrogenation catalyst is 5 weight percent copper on iron oxide or 5 weight percent copper in a copper-aluminum catalyst and the dehydration catalyst is H-mordenite. In this embodiment of the process of this invention it is preferred that the hydrogenation and dehydration catalysts are layered in a fixed bed and the reaction is carried out in the vapor phase and at a temperature in the range of about 300° C. to 350° C. and at a pressure in the range of about I to 30 atmospheres absolute, and the contact time of reactants is in the range of about 0.5 and 100 seconds.

The following examples describe the procedures used for the preparation of various catalysts employed in the process of this invention.

### EXAMPLE A

### Preparation of 5 Weight Percent Copper on Iron Oxide

Powdered and meshed iron oxide (100 g) of uniform particle size distribution of about 0.2 mm was dried at 120° C. in an oven under nitrogen atmosphere overnight and then cooled to room temperature. To this was added a solution of copper nitrate (17 g) in distilled water (100 ml). The resulting slurry was dried in an oven gradually heated to 110° C. (>2 hours, 10° C./m in.). The impregnated catalyst mixture was then calcined at 500° C. (6 hours, 1° C./min).

### EXAMPLE B

### Preparation of H-Mordenite Zeolite

H-Mordenite zeolite was prepared by calcination of ammonium form Mordenite at 500-550° C. for 4-8 hours. If the sodium form of Mordenite is used as a precursor, the sodium Mordenite is ion-exchanged to ammonium form prior to calcination.

### Gas Chromatographic (GC) Analysis of the Products

The analysis of the products was carried out by online GC. A three channel compact GC equipped with one flame ionization detector (FID) and 2 thermal conducting detectors (TCDs) was used to analyze the reactants and products. The front channel was equipped with an FID and a CP-Sil 5 (20 m)+WaxFFap (5 m) column and was used to quantify:

Acetaldehyde

Ethanol

Acetone

Methyl acetate

Vinyl acetate

Ethyl acetate

Acetic acid

Ethylene glycol diacetate

Ethylene glycol

Ethylidene diacetate

Paraldehyde

The middle channel was equipped with a TCD and Porabond Q column and was used to quantify:

CO₂

Ethylene

Ethane

The back channel was equipped with a TCD and Molsieve 5A column and was used to quantify:

Helium

Hydrogen

Nitrogen

Methane

Carbon monoxide

Prior to reactions, the retention time of the different components was determined by spiking with individual compounds and the GCs were calibrated either with a calibration gas of known composition or with liquid solutions of known compositions. This allowed the determination of the response factors for the various components.

### EXAMPLE 1

The catalysts utilized were a copper on iron oxide catalyst, T-4489 purchased from Sud Chemie and an H-mordenite zeolite prepared by replacing with hydrogen ions all but 500 ppm based on the weight of the zeolite of the sodium ions in a sodium aluminosilicate mordenite catalyst prepared in accordance with U.S. Pat. No. 4,018,514 or equivalent in which the ratio of silica to alumina is preferably in the range of from about 15:1 to about 100:1. A suitable catalyst is CBV21A available from Zeolyst International, which has a silica to alumina ratio of about 20:1.

In a tubular reactor made of stainless steel, having an internal diameter of 30 mm and capable of being raised to a controlled temperature, there are arranged 30 ml of 5 weight percent copper on iron oxide catalyst as top layer and 20 ml of H-mordenite as a bottom layer. The length of the combined catalyst bed after charging was approximately about 70 mm.

A feed liquid was comprised essentially of acetic acid. The reaction feed liquid was evaporated and charged to the reactor along with hydrogen and helium as a carrier gas with an average combined gas hourly space velocity (GHSV) of 2500 hr⁻¹ at a temperature of 300° C. and pressure of 100 psig. The feed stream contained a mole percent of acetic acid from about 6.1% to about 7.3% and mole percent of hydrogen from about 54.3% to about 61.5%. The feed stream was supplied to the hydrogenation catalyst (top) layer first such that the stream with hydrogenated acetic acid intermediates then contacted the dehydration catalyst layer. A portion of the vapor effluent from the reactor was passed through a gas chromatograph for analysis of the contents of the effluents. The acetic acid conversion was 65% and ethylene selectivity was 85%. Selectivity to acetone was 3%, selectivity to ethyl acetate was 2% and selectivity to ethanol was 0.6%. Carbon dioxide was relatively low; the measured selectivity to CO₂ of the acetic acid converted was 4%.

### EXAMPLE 2

The catalysts utilized were 5 weight percent copper on iron oxide prepared in accordance with the procedure of Example A and an H-mordenite zeolite prepared by replacing with hydrogen ions all but 500 ppm based on the weight of the zeolite of the sodium ions in a sodium aluminosilicate mordenite catalyst as noted above in Example 1.

The procedure as set forth in Example 1 was substantially repeated with an average combined gas hourly space velocity (GHSV) of 2500 hr⁻¹ of the feed stream of vaporized acetic acid, hydrogen and helium at a temperature of 350° C. and pressure of 100 psig. The resulting feed stream contained a mole percent of acetic acid of about 7.3% and mole percent of hydrogen of about 54.3%. A portion of the vapor effluent was passed through a gas chromatograph for analysis of the contents of the effluents. The acetic acid conversion was 8% and ethylene selectivity was 18%.

Generally speaking, selectivities to ethylene above 10% or so are highly desirable; it being appreciated that the other by-products such as ethanol or ethyl acetate can be re-cycled to the reactor along with unreacted acetic acid, while still other by-products can be re-processed or used for fuel value. Selectivities to CO₂ of less than 10% are desired, preferably less than 5%.

### COMPARATIVE EXAMPLES 1-5

These examples illustrate the reaction of acetic acid and hydrogen over a variety of catalysts wherein either no ethylene was formed and/or very low levels of ethylene was detected.

In all of these examples the procedure as set forth in Example 1 was substantially followed with the exception of using different catalysts as listed in Table 1. As summarized in Table 1, in all of these comparative examples only one single layer of catalyst was used. The reaction temperature and selectivity to ethylene are also tabulated in Table 1.

TABLE 1

| | | | | Mol % | |
|---|---|---|---|---|---|
| | | | Mol % | Acetic | |
| | | Reactor | H₂ | Acid in | |
| Reactor | | Temperature | in Feed | Feed | |
| Bed | Catalyst | (° C.) | Stream | Stream | Ethylene Selectivity |
| Single | 0.5%-1% | 250-350° C. | 54.2% | 7.3% | 0% |
| Layer | Pd on | | | | |
| | Carbon | | | | |
| Single | 1% Ru on | 250-350° C. | 36.8% | 7.3% | 0% |
| Layer | Carbon | | | | |
| Single | 2% Pt on | 350° C. | 34.3%-76.5% | 4.4%-7.3% | 0%-1% |
| Layer | Fe₂O₃ | | | | |
| Single | 2.58% Pd/ | 250-350° C. | 36.8% | 7.3% | 0%-0.5% |
| Layer | 5.05% Mo | | | | |
| | on SiO₂ | | | | |
| Single | 4.79% Cu | 400° C. | 35.2% | 7.5% | 0%-2.25% |
| Layer | on SiO2 | | | | |

In these examples various other products including acetaldehyde, ethanol, ethyl acetate, ethane, carbon monoxide, carbon dioxide, methane, isopropanol, acetone and water were detected.

Although the invention has been illustrated by certain of the preceding examples, it is not to be construed as being limited thereby; but rather, the invention encompasses the generic area as hereinbefore disclosed. Various modifications and embodiments can be made without departing from the spirit and scope thereof.

What is claimed is:

1. A process for selective formation of ethylene from acetic acid comprising: contacting a feed stream containing acetic acid and hydrogen at an elevated temperature with a first catalytic composition including a suitable hydrogenating catalyst in a first reaction zone to form an intermediate hydrogenated mixture; and reacting said intermediate mixture over a second catalytic composition which includes a suitable dehydrating catalyst and optionally a cracking catalyst in a second reaction zone to form ethylene.

2. The method according to claim 1, wherein the first and second reaction zones comprise respectively a first layer of the first catalytic composition and a second layer of the second catalytic composition in a fixed bed.

3. The method according to claim 1, wherein the first and second reaction zones are in separate vessels.

4. The method according to claim 1, wherein the selectivity to ethylene based on acetic acid consumed is at least 20 percent.

5. The method according to claim 1, wherein the selectivity to ethylene based on acetic acid consumed is at least 40 percent.

6. The method according to claim 1, wherein the selectivity to ethylene based on acetic acid consumed is at least 60 percent.

7. The method according to claim 1, wherein the selectivity to ethylene based on acetic acid consumed is at least 80 percent.

8. The process according to claim 1, wherein hydrogenation in the first reaction zone is carried out over a hydrogenating catalyst on a support, which catalyst is selected from the group consisting of copper, nickel, aluminum, chromium, zinc, palladium or a mixture thereof.

9. The process according to claim 8, wherein the support is selected from the group consisting of iron oxide, silica, alumina, titania, zirconia, magnesium oxide, calcium silicate, carbon, graphite and a mixture thereof.

10. The process according to claim 8, wherein the hydrogenating catalyst is selected from the group consisting of copper supported on iron oxide, copper-aluminum catalyst, copper-zinc catalyst, copper-chromium catalyst and nickel catalyst.

11. The process according to claim 8, wherein the hydrogenating catalyst is chosen from copper supported on iron oxide or copper-aluminum catalyst.

12. The process according to claim 1, wherein the second catalytic composition comprises a zeolite catalyst selected from the group consisting of H-mordenite, ZSM-5, a zeolite X and a zeolite Y.

13. The process according to claim 12, wherein the zeolite has a silica to alumina ratio (SiO₂/Al₂O₃) in the range of about 10 to 60.

14. The process according to claim 1, wherein said intermediate mixture comprises ethanol and ethyl acetate and said second catalytic composition includes a cracking catalyst.

15. The process according to claim 1, wherein the hydrogenating catalyst is copper on iron oxide and the dehydration catalyst is H-mordenite.

16. The process according to claim 15, wherein the loading of copper on iron oxide is in the range of about 3 weight percent to about 10 weight percent.

17. The process according to claim 15, wherein the loading of copper on iron oxide is in the range of about 4 weight percent to about 6 weight percent.

18. The process according to claim 1, wherein the hydrrogenating catalyst is copper-aluminum catalyst and the dehydration catalyst is H-mordenite.

19. The process according to claim 18, wherein the loading of copper on copper-aluminum catalyst is in the range of about 3 weight percent to about 10 weight percent.

20. The process according to claim 18, wherein the loading of copper on copper-aluminum catalyst is in the range of about 4 weight percent to about 6 weight percent.

21. The process according to claim 1, wherein hydrogenation and conversion to ethylene are carried out in the vapor phase and at a temperature in the range of about 200° to 375° C.

22. The process according to claim 21, wherein hydrogenation and conversion to ethylene are carried out in the vapor phase and at a temperature in the range of about 250° to 350° C.

23. The process according to claim 21, wherein said feed stream contains an inert carrier gas.

24. The process according to claim 21, wherein the reactants consist of acetic acid and hydrogen with a molar ratio in the range of about 100:1 to 1:100, the temperature of reaction zones are in the range of about 250° C. to 350° C., and the pressure of reaction zones is in the range of about 1 to 30 atmospheres absolute.

25. The process according to claim 21, wherein the reactants consist of acetic acid and hydrogen with a molar ratio in the range of about 1:20 to 1:2, the temperature of reaction zones are in the range of about 300° C. to 350° C., and the pressure of reaction zones are in the range of about 1 to 30 atmospheres absolute.
